(19) United States
(12) Patent Application Publication
(10) Pub. No.: US 2025/0268831 A1
(43) Pub. Date: Aug. 28, 2025

(54) COMPACT COMPOSITIONS COMPRISING ZINGIBERACEAE RHIZOMES FOR ACTIVE AND ADDITIVE APPLICATIONS AND MANUFACTURING METHOD THEREOF

(71) Applicant: ALEXIA C. HARVEY, Portmore (JM)

(72) Inventor: ALEXIA C. HARVEY, Portmore (JM)

(21) Appl. No.: 17/849,674

(22) Filed: Feb. 25, 2024

(51) Int. Cl. A61K 9/16; A61K 36/9066; A61K 36/9068

(52) U.S. Cl. CPC A61K 9/1652; A61K 36/9066; A61K 36/9068

(57) ABSTRACT

Compacts may include: a first layer comprising turmeric, ginger, and/or starch, and may be configured to release constituents in an aqueous environment, optionally having a granule, pellet and/or tablet morphology. Such compacts may have a total mass of no more than 13 g, whereby each layer may gave a total mass of no more than 13 g. Such compacts may have a hardness above 3 kg and/or a friability of 0.0001 to 7′%. The layers may be positioned at a 0 to 90° angle relative to the pressing direction. Such compacts may have a thickness in a range of from 0.0001 to 12 cm, whereby each layer may have a thickness in a range of from 0.0001 to 12 cm.

### FIELD

An aspect of the invention relates to compositions configured for use as dosage forms or condiments, and methods of production of the invention.

### BACKGROUND OF INVENTION

Compacts are systems that have a vast range of applications. Functions include, but are not limited to, therapeutic (in, e.g., dosage forms), cleaning, or sanitising, and the seldom seen application where a compact may contain a part or complete solute composition of a beverage. Examples of such systems are described in WO 2015/171445 A1, U.S. Pat. No. 5,783,540, and WO 2013/084072 A2.

Other types of systems tend to be less chemically stable, bulkier, and/or present an inconvenience for transport. These include liquids, semisolids, or some with solid constituents of varying sizes such as tea bags, which if not handled carefully the bags may easily become ruptured. Like these systems, the constituents of compacts can be synthetic or of natural origin.

Turmeric and ginger are two examples of natural constituents. They are erect perennial herbs generally grown for their rhizomes. Both plants are of the Zingiberaceae family, and are cultivated in tropical and subtropical countries. The characteristic flavour and aroma of these rhizomes result in them being highly regarded in culinary applications to enhance the organoleptic appeal of the preparations. These include curry spices, foods, and teas and other beverages. Examples of turmeric and ginger application as condiments are described in WO 2007/137297 A2 and CN 1,390,477 A.

The utilisation of the turmeric and ginger plants is not limited to their application as adjuncts/additives/condiments. They are also applied as therapeutic agents and/or functional foods and have been done so for centuries. Examples of turmeric and/or ginger rhizomes, or derivatives, being applied in products as active agents (therapeutic agents and/or functional foods) are described in U.S. Pat. Nos. 10,772,926 B2, 2,009,226,550 A1, CN 100,391,532 C, KR 102,219,923 B1, CN 112,369,536 A and KR 20,090,039,829 A. Folklore and modern research have shown medicinal applications to include their use as anti-inflammatory treatments, and antinociceptive agents for treating acute, chronic, or acute and chronic pain. Turmeric has also been found to have anticancer, anticoagulant, antioxidative, and antimicrobial properties, while ginger has been proven to be an anti-nauseant. As such, both have been utilised in nutraceutical preparations, including solid dosage forms for oral administration. This category of dosage form and route of delivery are those preferred in general.

Solid dosage forms include granules (aggregates of powders), pellets, tablets and capsules. The most frequently administered of these are tablets followed by capsules, owing to their allowance for ease of self-administration, a wide range of possible drug administration and dosage adjustment, and the possibility to terminate treatment once untoward effects are detected. Tablet production methods vary in both complexity and cost; of them all, direct compression (DC) is often the most favoured. Other methods are more expensive as they require special equipment, the addition of a liquid, or/and special handling with the application of a liquid and/or the drying of the formulation constituents. DC production is also advantageous as it ensures that drugs that are highly water soluble do not become readily predisposed to hydrolysis. In addition, this method of production tends to be faster. Unfortunately, very few substances including drugs, also called active pharmaceutical ingredients (API), have been found to be amendable to the DC process, primarily as a result of their inherent poor fluidity and compressibility. Dosage formulations for compaction therefore frequently require the conversion of powders to granules to improve their overall fluidity and compressibility. DC excipients may also be utilised to allow for the possible omission of granulation, wet or dry.

### SUMMARY OF THE INVENTION

A compact composition may be configured for the release of its constituents including turmeric, ginger, or turmeric and ginger, for applications including nutraceutical, preservation, and/or culinary application.

The compact may comprise (i) turmeric, (ii) ginger, (iii) starch, or a mixture of (iv) any two, or (v) all three, preferably each in its dried powdered form. For the turmeric and ginger powders, this is preferably of the rhizomes of the plant. The composition may comprise a greater weight percentage (wt. %) of the turmeric constituent, relative to that of the ginger constituent. The composition may comprise a greater wt. % of the ginger constituent, relative to that of the turmeric constituent.

The compact may be a constituent of a secondary (resulting from a primary first product) product comprising a composition of a final state of matter that is solid, semisolid or a liquid. The secondary product may be a dosage form, a condiment, a food, and/or a beverage.

The compact may comprise of one or more layers, and the positioning of the layers may vary. One or more layers may be at varying angles from two or more directional reference planes. The layers may be unevenly distributed, that is not regularly or consistently arranged, within another layer, or layers may be arranged in a regular fashion; here regular implies being arranged in a constant or fixed pattern, e.g., stacked one above another with 0 mm spacing length between consecutive layers.

The inventive compact may comprise a condiment for application in a food and/or beverage. The compact may also comprise a dosage form comprising therapeutically effective amounts of turmeric and/or ginger constituents. Preferably, the dosage form is a solid dosage form inclusive of granules, pellets, or tablets. More preferably, the dosage form is a tablet. Tablet pharmaceuticals generally comprise no more than 25 wt. % of active ingredients due to the tendency of the actives to have low compressibility properties. The turmeric and/or ginger constituents may constitute more than 25 wt. % of the composition of the dosage form compact. The compact and/or a secondary product comprising the compact, may be administered as, for example, an anticancer, anti-inflammatory, antinociceptive agent, and/or antinauseant. Inventive compacts may therefore be useful in aiding in the relief of conditions including headaches, arthritic pain, post-surgical pain, motion sickness or nausea, or menstrual cramps coupled with vomiting.

Compacts comprising turmeric and/or ginger may exhibit desirable hardness, friability, and/or disintegrating properties. possibly indicative of needing no binder and/or disintegrant in the manufacturing of the compacts. Desirable hardness, friability and disintegration implies a hardness that allows the invention to disintegrate within chosen limits, a friability with a upper limit of 1% mass loss, and disintegration with a upper limit within a chosen limit, e.g., a dispersible compact which disintegrates within 3 minutes, respectively. Compacts comprising turmeric and/or ginger may also exhibit properties that permit the easy removal or ejection of the compact from the die after its formation by the compression of its constituents. Accordingly, inventive compacts may avoid the use of a lubricant in the compact manufacturing process.

### BRIEF DESCRIPTION OF THE DRAWINGS

The following drawings are several exemplary illustrations within the scope of the invention. Like reference numbers indicate the same or similar elements of the invention. Broken lines are illustrative of periphery surfaces of embedded layers (within one or more layers of the compact) that are not presented or visible on the outer periphery (external) surface of the invention, once the structure of the compact remains intact, i.e., the structure of the compact has not been jeopardised disturbed. Here jeopardised/disturbed implies damaged or ruptured.

FIG. 1 shows a diagrammatic upper front right perspective view representation of an exemplary compact, in its undisturbed state, comprising at least one layer according to the present invention.

FIG. 2 shows a diagrammatic upper front right perspective representation of an exemplary compact, in its undisturbed state, comprising at least two layers according to the present invention.

FIG. 3 is a diagrammatic representation of a upper front right perspective view of an exemplary compact, in its undisturbed state, comprising at least three layers according to the present invention.

FIG. 4 shows a diagrammatic representation of a top plan view of an exemplary compact, with an ovoidal shape, in its undisturbed state, comprising at least one layer according to the present invention.

FIG. 5 shows a bottom front perspective view, including a theoretical internal view, of an exemplary compact with the cross-sectional area visible, comprising a second layer of the compact that is completely encompassed/enveloped and centralised within a first layer that is the outer surrounding layer of the compact.

FIG. 6 shows a bottom front perspective view. including a theoretical internal view, of an exemplary compact with the cross-sectional area visible, comprising a completely encompassed/enveloped centralised layer, completely encompassing at least one layer, within the outer surrounding layer of the compact.

FIG. 7 shows a bottom front perspective view, including a theoretical internal view, of an exemplary compact with the cross-sectional area visible, comprising multiple completely encompassed/enveloped dispersed layers, within the outer surrounding layer of the compact.

FIG. 8 shows a bottom front perspective view, including a theoretical internal view, of an exemplary compact with the cross-sectional area visible, comprising partially encompassed/enveloped dispersed layers, within the outer surrounding layer of the compact, with the partially encompassed layers being visible as two-dimensional shapes on the outer periphery surface of the outer surrounding layer.

FIG. 9 shows a bottom front perspective view, including a theoretical internal view, of an exemplary compact with the cross-sectional area visible, comprising partially encompassed/enveloped dispersed layers, within the outer surrounding layer of the compact, with the partially encompassed layers being visible as protrusions on the outer periphery surface of the outer surrounding layer.

FIG. 10 shows a schematic representation of several exemplary methods of manufacturing the inventive compacts, within the scope of the invention.

### DETAILED DESCRIPTION

The present invention can provide formulation mixtures for compacts and/or compact mixture systems, optionally for the application of components comprising turmeric, ginger, starch or any mixture thereof, in various combinations, in generally recognised as safe (GRAS) dosage forms or condiment application.

It is not typical of materials in their native unmodified state (without physical or chemical modification processing) to comprise binding capacity to allow for the formation of a compact from dry powdered constituents. This is also not typical of synthetic active pharmaceutical ingredients. Synthetic implies the material is artificial or not of natural means. Here, ‘natural’ means being of an organism, such as but not limited to plants, animals, fungi, and/or bacteria.

Likewise, turmeric and ginger rhizomes, undried or dried, do not comprise binding capacity to allow for the formation of the inventive compacts. The turmeric and/or ginger may be cultivated and/or sourced from Jamaica, India, Sri Lanka, America, Brazil, and/or Madagascar. This is also the same, for turmeric rhizome powder or ginger rhizome powder, produced by drying the rhizome to a moisture content that may be as low as 4.5, 4.8, 5.7, 6, 6.2, or 7.5% and as high as 14, 13, 11.5, 10, 7.5, 6, 5.3, or 4.8%, and milling the rhizome, without the powder being subjected to further processing. The mixing of the turmeric powder and the ginger powder, without further processing of the resultant powder mixture, likewise, will not result in the powder mixture comprising binding capacity to allow for the formation of the inventive compacts. Further processing may include (i) granulation, or (ii) application of a compressional pressure. Granulation implies the conversion of a powder to larger multi-particle entities/agglomerates, called granules, with a general outcome of an improved fluidity and compressibility of the material.

Unmodified starch is a natural material. Traditional sources include corn (maize), rice, potato, and wheat. Untraditional sources include the tubers of Dioscorea sp. yam and dasheen. During the extraction of native starch. agglomerates (produced from starch particles interacting with a liquid) may be formed. To get the starch to its native powdered form/state, the agglomerates are milled grounded. Starch in its native state, is not known to comprise binding capacity to allow for the formation of compacts. Here, starch refers to one or more types of starch; therefore, the term ‘starch’ can be used to refer to a collection of starch or to substitute for the word starches.

In addition, native starch, being subjected to compressional pressure, typically will not result in the formation of compacts, or will result in the formation of compacts that are likely to rupture without the application of an external force. The inability to successfully produce stable compacts is owed to the inherent limitation of poor plasticity of native starch in general, thereby commonly hindering its utilisation as a direct compression (DC) binder. The main function of a DC binder is to allow dry powdered formulations, of which the DC binder is a constituent, to form stable compacts, without the addition of a liquid. The mixing of (i) native starch and (ii-a) turmeric powder or (ii-b) ginger powder. or (iii) turmeric powder and ginger powder, without further processing of the resultant powder mixture, likewise, will not result in the powder mixture comprising binding capacity to allow for the formation of compacts. Further native starch processing, such as (i) granulation and/or (ii) the application of compressional pressure, may take native starch from its natural state to allow for the formation of compacts. An example of native starch that can result in the formation of a compact with further processing only involving the application of compressional pressure, i.c., is amendable to DC is described in U.S. Pat. No. 5,453,281 A.

The present invention is based in part on the discovery that turmeric powder and or ginger powder may be configured to function as, at least a portion of, an adjunct constituent of the inventive compacts. Constituents of the inventive compacts may be of powder, pellets, granules and/or tablet morphology. The invention compacts may be pellets, granules and/or tablets. The turmeric and/or the ginger may also possess plurality function including being an adjunct. Examples of their plurality function may be, (i) the turmeric and/or the ginger functioning as a therapeutic agent and adjunct, and/or (ii) the turmeric and/or the ginger functioning as no less than two adjuncts, in the inventive compacts. Here therapeutic agent implies a substance utilised to treat/ease mitigate a disease. Here, a functional food, on the other hand, implies a food that beyond its nutritional value can promote optimal health and aid in reducing the risk of disease (disorders of structure or function of an organism). An adjunct however is a constituent that is not an essential part thereof or can be said to be a supplementary part of the invention. Specifically, in addition to being therapeutic agents and/or functional foods, the turmeric and/or the ginger constituents may function as, at least a portion of, for example, binders, lubricants, colourants, preservatives, flavourings, disintegrants, and/or multifunctional adjuncts. Their multifunctional additive/adjunct properties may allow the turmeric and/or the ginger to contribute to properties of the compact such as the structural integrity, appearance, or content release rate of the compact, as well as, e.g., the induced organoleptic properties of a secondary product that comprises the inventive compacts. Within the present invention. (i) turmeric powder and (ii) ginger powder, may be referred to as ‘the turmeric’ and ‘the ginger’ respectively, herein.

The present invention is also based in part on the discovery that natural dasheen (Colocasia esculenta) starch may be configured to function as a multifunctional compact additive, with several properties allowing for it to supplant, at least a portion of, a binder, e.g., a DC binder, a disintegrant, a filler (used to bulk up/make up the final quantity of a composition which, prior to the addition of the filler, was of a quantity smaller than that desired), and/or a customary multifunctional additive/adjunct comprising binding, disintegrating and/or compact filling properties, of the inventive compacts.

The inventive compacts may comprise any one, any two, or all three of the turmeric, the ginger, or the starch constituents. The quantities (w/w) of the turmeric, and/or the ginger may be of any region of the rhizome of the respective plant type. The turmeric, and/or the ginger wt. % may be of the compact and/or of individual layers (independently) in amounts of, e.g., at least 0.25, 1.5, 3, 6, 10, 20, 25, 35, or 40 wt. % and/or to up to 100, 90, 35, 20, 10, 3, 2, or 1 wt. %, relative to a total mass of at least, e.g., 0.025 to 13 g. Within the inventive compacts, the turmeric, the ginger and/or the starch, having been subjected to compressive stress during compact production, may be suitable to functionally supplant, or at least a portion of, a customary binder. For example, the turmeric, the ginger, and/or starch may supplant, at least a portion of, a DC binder. The presence of the turmeric, the ginger and/or starch may therefore result in no further binder being required for the formation of the inventive compacts. Within the inventive compacts, turmeric and/or the ginger may also be suitable to functionally supplant, at least a portion of. e.g., a customary lubricant and/or a customary glidant, while starch may also supplant, at least a portion of, e.g., a customary filler, and or a customary disintegrant. Customary implies used traditionally or untraditionally, in industry, Such compacts, comprising turmeric and/or ginger, may require no further lubricant and/or glidant. If compacts comprise starch, they may require no further filler and/or disintegrant. Other native nontraditional starch that may exhibit the afore mentioned starch adjunct properties in the inventive compacts, include, e.g., some Dioscorea sp. yam starch, and/or Irish potato (Solanum tuberosum) starch. Within the present invention, native starch of dasheen, Dioscorea sp. yam and/or Irish potato, may be referred to as the starch, herein.

Preparation of the turmeric, the ginger and/or the starch raw materials of the inventive compacts, may initially involve drying their sources, e.g., turmeric rhizomes, ginger rhizomes, and one or a plurality of sources (e.g. dasheen tuber) of starch, by air, sun, vacuum, conventional oven, or by a combination thereof. The sources may be dried whole, and/or as smaller masses produced from slicing up the sources, e.g., dicing a dasheen tuber into smaller masses. The process also involves grounding milling shredding of the whole and/or sliced up pieces, which may precede the drying process. The desired resultant product morphology from milling is a powder. The shredded composition may then be sieved. The composition may then undergo varying forms of final preparation such as granulation, compaction, encapsulation, spray drying, or the like to attain a final product of the inventive compacts and/or a secondary product inventive compact. Preparation of the turmeric, the ginger and the starch raw materials of the inventive compacts, may also involve sanitising (cleaning and sterilising) the sources, at least once, at any stage, preferably before the milling of the sources, to minimise mass loss.

The inventive compacts may further comprise additional natural constituents and/or synthesised constituents. An additional natural constituent, or a synthetic constituent, in the compacts, may function as an adjunct, a therapeutic agent and/or functional food. Examples of additional natural constituents are coffee, chocolate, starch, moringa, garlic, sugar, stevia, cinnamon, mint leaves. lemon grass and/or green tea leaves. Additional natural ingredients that may be applied as adjuncts in the compacts are native starches from other sources. Examples of other forms of native starch include rice and corn starch. Synthetic constituents, that may be admixed for their adjunet properties, include but are not limited to, aspartame, saccharin, tale, magnesium stearate, benzoate salts, and/or microcrystalline cellulose. These natural and synthesised adjuncts that may be applied, are suitable for use as excipients in pharmaceuticals and/or nutraceuticals. Examples of natural constituents with active properties that can be added to the inventive compacts are moringa and cinnamon for their functional food and/or therapeutic properties. Synthetic constituents that may be applied as actives, in conjunction with the inventive compact and/or added to the compacts, are anticancer drugs, nonsteroidal anti-inflammatory drugs (NSAIDs) such as asprin (acetylsalicyclic acid), naproxen, and toradol (ketorolac); paracetamol (scientific name acetaminophen, which is not an NSAID due to its low anti-inflammatory properties), ibuprofen, bismuth subsalicylate (used for nausea, vomiting, and as a antidiarrheal), and morphine (an opioid agonist painkiller that can have side effects including vomiting and nausea). Advantages that may be had with these actives being administered along with the turmeric and/or the ginger are that there may be an increased therapeutic effect, and/or counteraction of adverse effects of the synthetic actives, particularly with the presence of the ginger.

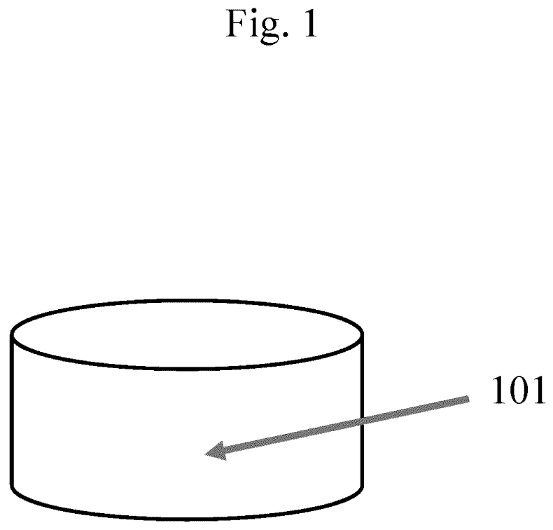

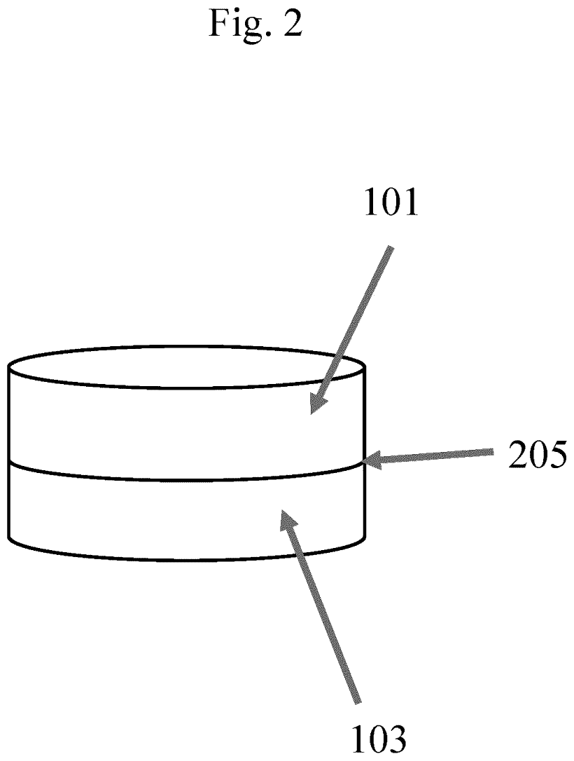

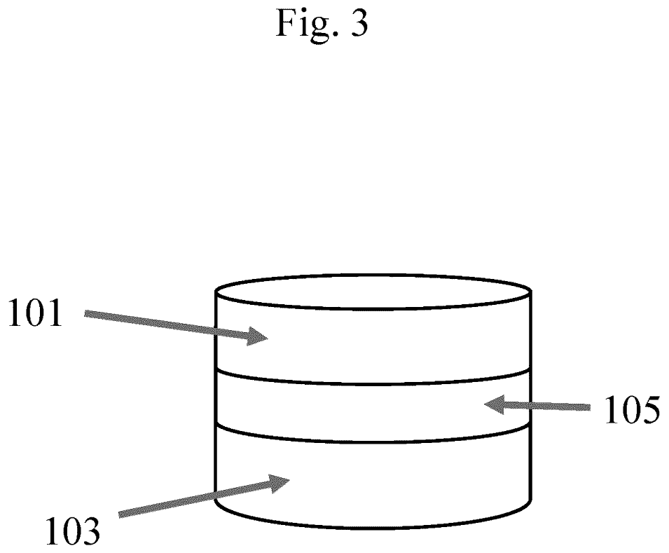

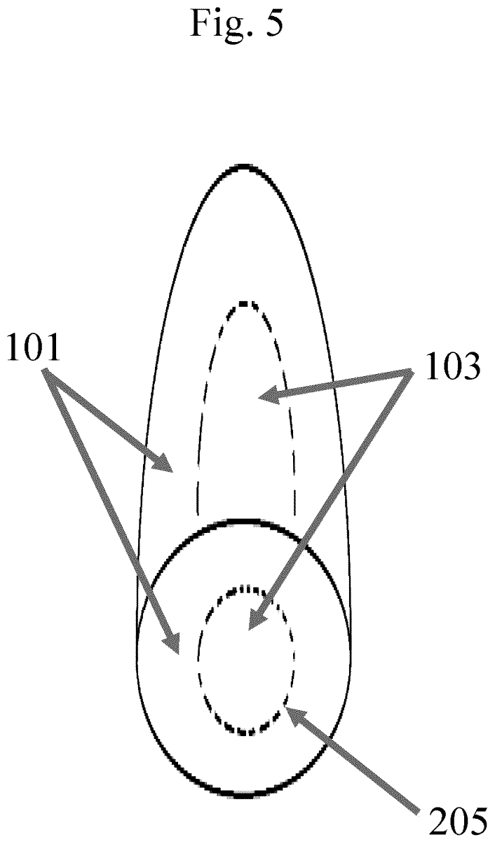

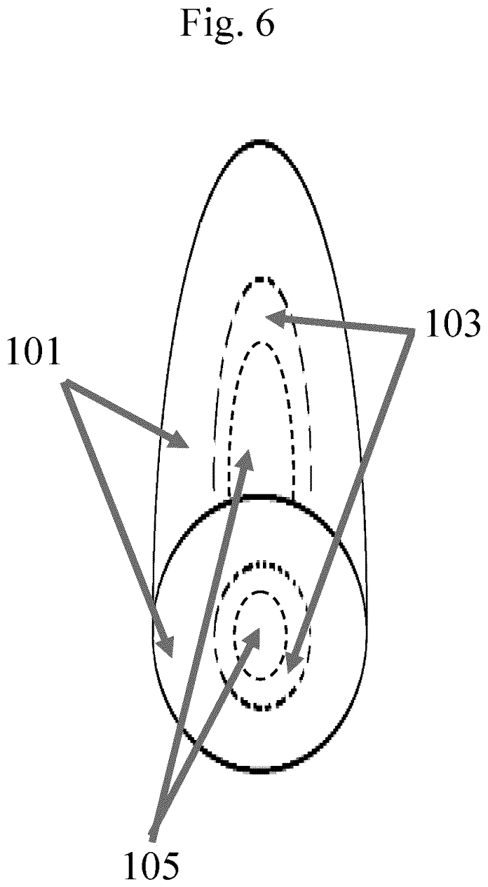

The inventive compacts may have a first layer, a second layer, a third layer, a fourth layer, etc., up to, for example, 12 layers, 11 layers, 9 layers, or 3 layers, as desired. The layers may not necessarily be ordered as named, i.e., for example, a second layer may not follow, in any direction, a first layer, or be in immediate contact with a first and/or a third layer. Such compacts, in their undisturbed state, may have, e.g., only one, two, three or more layers, and these layers are also visible layers, e.g., as seen in FIGS. 1 and 4, FIG. 2, FIG. 3 and FIGS. 8 to 9, respectively. The layers may be positioned at a 0 to 90° angle from an orthogonal plane to a pressing direction, e.g., at least at a 4, 5, 11, 15, 30, or 45° and/or up to 90, 88, 84, 78, 75, 60, 55, 45, or 30°. For example, FIGS. 2 and 5 have layers 101 and 103, and FIGS. 3 and 6 have layers 101, 103, and 105, arranged in a stacked pattern, with the layers of FIGS. 2 to 3 positioned at a 0° angle from a horizontal plane, where the chosen reference horizontal plane is that parallel to the layers, running from left to right of the top perspective view shown in FIGS. 2 to 3. The layers of FIGS. 5 to 6 are positioned at a 90° angle from the said horizontal plane.

The present invention may have a first layer and/or second layer that may partially or completely encompass or have embedded, at least one layer. For example, 1, 2, 3, 4, 5, 6, 7, or 8 layers being partially and/or completely embedded in 1, 2, 4, 10, or 12 layers, but typically no more than 8, 3, or 1 layer being partially and/or completely embedded in 5, 4, 2, or 1 layer. Examples of layers being completely/fully embedded are shown in FIGS. 5 to 7, with the embedded layer 103 in the outer surrounding layer 101, and in FIG. 6 the completely embedded layer 105 in layer 103, represented by broken lines. Within the body of the inventive compacts, embedded layers may be centralised. Examples are FIG. 5 with the centrally located layer 103, and FIG. 6 with layer 105 being completely embedded in the centralised layer 103, which is embedded in the outer surrounding layer 101.

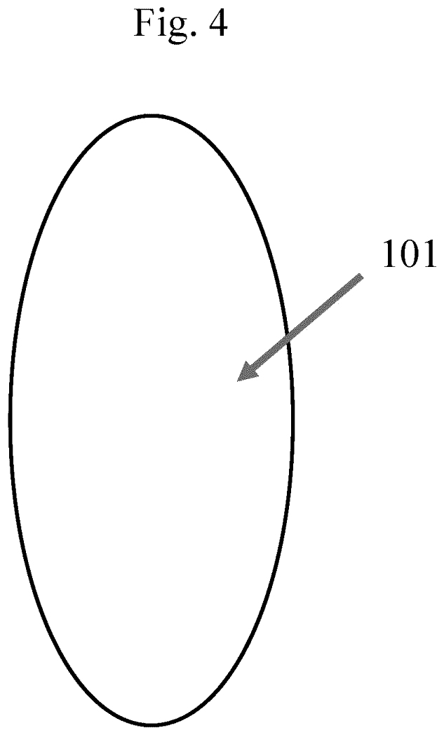

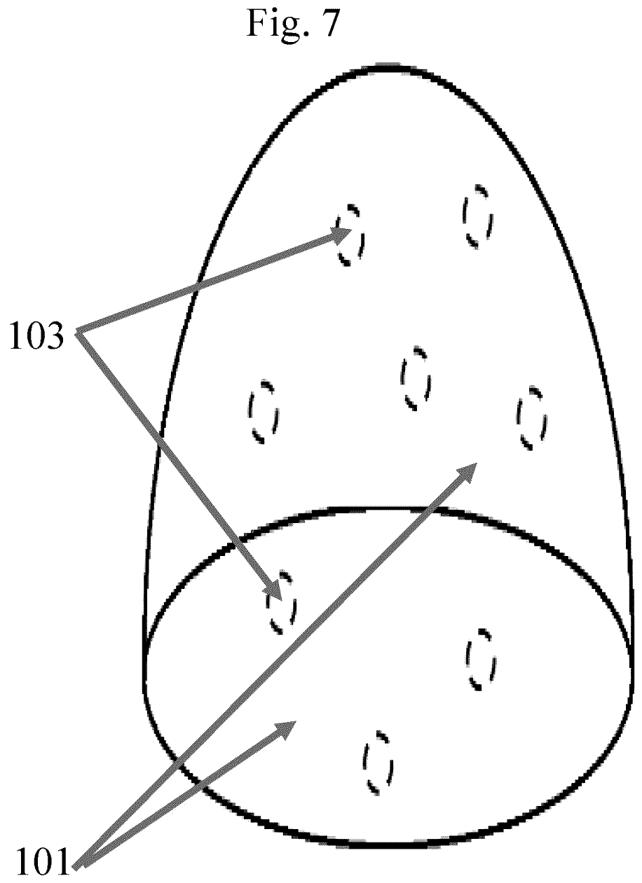

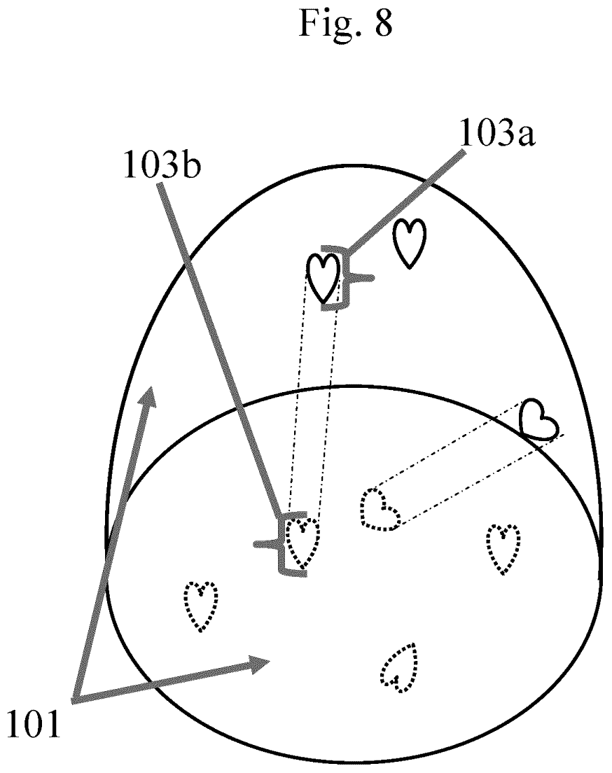

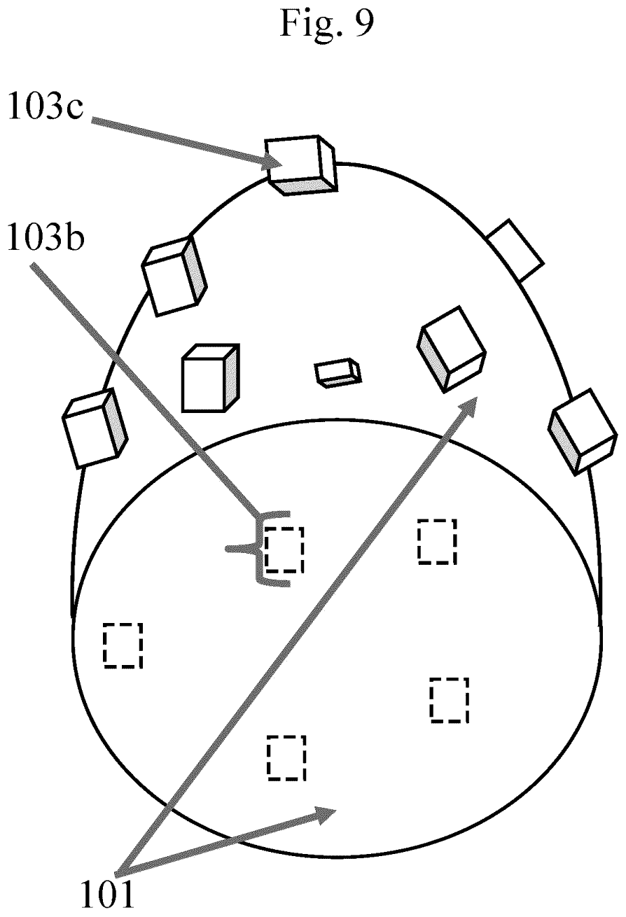

The moulded shape of the inventive compacts, and/or its layers may vary. Within the present invention, the shape of the compact and/or layers may be, but is not limited to oval, circular, cylindrical, star-shaped, heart-shaped, rectangular etc. In FIGS. 1 to 3, the compacts are cylindrical in shape and in FIGS. 4 to 9, the compacts have an ovoidal shape. In FIGS. 5 to 6, the embedded layers are ovoidal. The embedded layers in FIG. 7 are circular; those in FIG. 8 are cylindrical, comprising heart-shaped outer top and inner bottom periphery surfaces, e.g., that of layer 103 with outer top and inner bottom periphery surfaces being identified as 103a and 103b, respectively; and the embedded layers of FIG. 9 are rectangular. The periphery or exterior surfaces of a layer is an outer edge of the respective layer. Embedded layers of the inventive compacts may extend from within the compact to an inner and/or outer periphery surface of at least one layer, e.g. 1, 2, 3, 7, or 10 layers may extend to the inner and/or outer periphery surface of 1, 2, 4, 9, or 13 layers, but typically no more than 5, 3, or 1 layer extending to the inner and/or outer periphery surface of 6, 9, 3, or 1 layer. Examples are layer 103 in FIGS. 8 to 9. Layers that extend to the outer periphery surface, i.c., partially embedded layers, of one or more layers, may be visible as at least one two-dimensional shape on the outer periphery surface of the inventive compacts, e.g., the heart-shaped outer periphery surface of layer 103 in FIG. 8. The compact may further comprise one or more layers that extend to the outer periphery surface of one or more layers and is visible on the outer surface of the compact as three-dimensional protrusions, e.g. 103c of FIG. 9.

The inventive compacts may comprise a plurality of layers, as low as, e.g., 5, 6, 7 or 9 up to 10, 8, 6, 5, 3 or 2 layers, but typically no more than 8, 7, or 5 layers within 5, 3, 2, or 1 layers, that are arranged in a stacked pattern such as those of FIGS. 1 to 3 and/or 5 to 6. Inventive compacts may alternatively or additionally comprise a plurality of scattered/dispersed (randomly distributed) layers embedded within at least one layer, i.e., one or more shapes of compacts being encompassed within one or more partially and/or completely surrounding layers; e.g., a plurality of scattered layers, such as 2, 3, or 5 scattered layers up to 10, 8, 6, 5, 4, 3 or 2 scattered layers, partially and/or completely surrounded by one or multiple layers. Examples are FIGS. 7 to 8, with completely embedded scattered layers within layer 101, one of which is identified as 103. The scattered layers of the inventive compacts may be interspersed between non-scattered layers, e.g., in an alternating pattern, or in some kind of sequence, such as scattered-scattered-non-scattered-scattered-scattered-non-scattered . . . ; or 2, 3, 4 or more stacked layers—2, 3, 4 or more scattered layers 2, 3, 4 or more stacked layers; or the like.

Inventive compacts may be constituents of a secondary inventive compact, such as a layer that underwent precompression to form a constituent compact which is to be embedded partially and/or completely within one or multiple layers. The constituent inventive compact may also be at least one of a plurality of stacked and/or scattered layers.

The thickness, in a longest dimension in a orthogonal plane to a pressing direction and/or at the perimeter/circumference and/or centre, of the inventive compact, in any directional plane when measured horizontally, vertically, and/or diagonally, may be in a range of from 0.0001 to 12 cm, e.g., at least 0.0001, 0.0002, 0.0005, 0.0018, 0.0025, 0.005, 0.013, 0.025, 0.033, 0.05, 0.067, 0.075, 0.1, 0.15, 0.2, 0.25, 0.3, 0.33, 0.4, 6, 7 or 10.5 cm and/or up to 12, 10, 8, 7, 6, 5, 4, 3, 2, 1, 0.75, 0.67, 0.5, 0.33, 0.25, 0.2, 0.175, 0.15, 0.125, 0.12, 0.11, or 0.1 cm. Each layer. independently, at the perimeter/circumference and/or centre, may have a thickness, in any directional plane and/or in a longest dimension in an orthogonal plane to a pressing direction, in a range of from 0.0001 to 12 cm, e.g., 0.0001, 0.00025, 0.0005, 0.001, 0.0025, 0.005, 0.017, 0.025, 0.033, 0.5, 0.067, 0.075, 0.1, 0.15, 0.2, 0.25, 0.3, 0.33, 0.47, or 0.53 cm and/or up to 12, 10, 8, 7, 6, 5, 4, 3, 2, 1, 0.75, 0.67, 0.5, 0.33, 0.25, 0.2, 0.175, 0.15, 0.125, 0.12, 0.11, or 0.1 cm, when measured horizontally, vertically, and/or diagonally. The spacing length or distance between any two layers of, e.g., 2, 3, 4, 6, 7, 8 or 10 layered compacts, from a first point to a second point between the two layers, may range from 0.00001 to 120 mm, e.g., at least 0.001, 0.0025, 0.01, 0.03, 0.05, 0.064, 0.079, 0.09, 1, 2.6, 3.7, 8, 9, or 10 mm and/or up to 11.87, 11.6, 10, 9.8, 7.7, 2, 0.13, 0.01, or 0.003 mm. The spacing may be air, a carrier, a barrier layer, such as a cellulose or digestible (or otherwise dissolvable) film, and/or a layer lacking ginger, turmeric, and or starch, particularly lacking all three. The barrier layer may completely or partially occupy the space between two or more (i) layers that it encompasses partially or completely, and/or (ii) layers that are adjacent to it. The spacing between two layers of the inventive compact may comprise spaces as small as that between two particles (interparticle space, i.e., essentially unspaced or directly contacting layers) or the sum of the interstitial spaces between particles of the periphery surface of 2 consecutive layers. One or more layers may be equally and/or unequally spaced from one or more layers, e.g. 0.0 to 0.000001, 0.00003 to 0.05, 0.0001 to 3, and/or 1 to 100 mm spacing between or among layers, but typically no more than 0.00001 to 10, 0.0005 to 3, and/or 0.0002 to 50 mm spacing. An illustration of there being no space between two consecutive layers of the inventive compacts may be, e.g., the inner periphery surfaces of layers 101 and 103 of FIGS. 2 and 5 having a spacing length of 0.00 mm, resulting in what can be seen as a layer division line being formed, labelled as 205. Here the layer division line constitutes the spacing distance between 2 layers; the thicker the line the greater the spacing length. The layers of the compacts may be distinguishable based on properties including but not limited to colour, constituent weight percent, thickness, hardness, dissolution rate, and/or particle/grain type and/or size of the content of the layer.

The inventive compacts are of a total mass that may be as low as 0.02, 0.10, 0.25, 0.275, 0.30, 0.075, 0.5, 1.0 g, etc., and/or as high as 13, 12, 10, 6 or 3 g. The mass of each layer may be as low as 0.004, 0.01, 0.025, 0.06, 0.075, 0.10, 0.13, 0.25, 0.275, 0.285, 0.324, or 4.015 g, and as high as, e.g., 13, 9, 9.8, 7, 3.001, 3, 2.75, 2.005, 2, 1, 0.155, 0.13, or 0.03 g. The compacts of the present invention may comprise an upper limit of 100 wt. % natural ingredients, relative to the total mass of the compact. Such natural ingredients may be modified by chemical and/or physical means, such as pressure, outside of their normal state, even imparting advantageous properties. One or more layers of the compacts may also comprise an upper limit of 100 wt. % synthesised constituents, relative to the total mass of the respective layer. The inventive compact and/or at least one layer, may comprise, e.g., no less than 20, 22, 24.6, 30, 31.1, 33, 34.5, 38, 43.8, 47.2, 50, 51.8, 52.3, 55, or 72 wt. % and/or up to 88, 82.5, 80, 77.5, 72, 66, 52.3, 51, 45.5, 36, 32 or 30 wt. %, of natural constituents, and, e.g., at least 0.03, 0.033, 0.043, 0.05, 0.1, 70, or 71 wt. % and/or up to 78, 77.5, 72, 62.3, 57.5, 49, 45.5, 38.7, 37.5 or 30 wt. % of synthesised constituents. One or more layers may comprise of at least 0, 5, 15, 25, 35, 85, 90, 93.7 or 95 wt. % and/or up to 110, 107.5, 106.0, 100, 95, 50, 35, 20, 18.5, 4.5 wt. %, of turmeric, 0, 2, 5.5, 8, 13, 15, 20, 85, 92.5, 93.7 or 90 wt. % and/or up to 110, 107.5, 106.0, 100, 95, 50, 35, 20, 18.5, 4.5 wt. %, of ginger, and/or at least 0, 2.5, 4, 5, 15, 25.5, 70, 85, 90, 93.7 or 95 wt. % and/or up to 100, 95, 65, 35, 20, 18.5, 4.5 wt. %, of starch, relative to the target total mass of each respective layer.

The inventive compacts may have a averaged hardness upper limit of 30 kg, e.g., at least 0.4, 0.42, 0.84, 0.9, 0.97, 1.5, 1.59, 1.7, 1.75, 1.83, 1.95, 2.05, 2.33, 2.42, 2.5, 2.67, 2.71, 3, 3.11, 3.21, 3.34, 3.6, 5, 7, 8 or 10 kg and/or up to 50, 40.5, 40, 30, 20, 15.6, 12.24, 12, 11.98, 11.61, 11, 10, 9.01, 7.3, 6.4, 2.5, or 1.6 kg. The averaged friability of the inventive compacts may be within a range of from 0.44, 0.5, 0.68, 0.9, or 1% to no more than 0.9, 1, 1.2, 1.5, or 1.9, 2.5, 3.33, 3.67, 4, 4.5, 5, 6, or 7%, e.g., 0.001, 0.05, 0.1, 0.13, 0.18, 0.22, 0.28, 0.4, 0.6, 0.7, 1.5, 1.9, 2.1, 4.5, or 6.9% and/or up to 6.95, 6.7, 6.37, 5.43, 4, 3, 2.77, 1, 0.9, 0.21, 0.14, or 0.1%. Hardness or crushing strength of a compact is the load needed to crush the compact. Friability, like hardness, assesses the physical stability of a compact. Friability is a measure of the ability of the compact to withstand abrasion with handling, during packaging and or shipping. These parameters are useful as they may have a significant influence on the disintegration of the invention. The hardness and friability values were favourable as they may result in complete disintegration being achieved in at least 0.00011, 0.0009, 0.01, 0.016, 0.2, 0.3, or 0.4 and/or up to 0.04, 0.09, 0.05, 0.067, 0.17, 0.3, 0.5, 1, 2.5, 5, 10, 15, 24, 36, or 49 hours.

The compacts of the present invention are configured to release their constituents in an aqueous environment, in a range of from 0 to 100° C. at ambient atmospheric pressure. The physicochemical properties of one or more constituents may influence the release rate of one or more constituents, from one or more layers, within time 0 to t (t represents the time within which there is complete release of all constituents from the compact). Examples of these physicochemical properties may include projected particle size (e.g. the turmeric, and/or the ginger comprises a projected particle size of at least in a range of from 1.5 to 145 μm), particle size range, moisture content, solubility, intra and/or inter-particle interactions, constituent distribution within the structure, and/or binding capacity. Disintegration of the compacts and/or at least one layer may take place within a range with a lower limit of, e.g., 0.0008, 0.001, 0.0011 hours to an upper limit as high as 49, 36, 24, 18, 12, 8, 4, 3, 0.5, 0.4, 0.3, 0.25, or 0.05 hours, in an aqueous environment. The inventive compacts may be configured such that a limiting factor for disintegration is a release rate of constituents from at least a first layer, or a first and second layer, or more than three layers.

The inventive compacts may be dosage forms that comprise, e.g., pellets, granules, and/or tablets. Here a pellet has a spherical or globe shape and a diameter less than or equal to 2.5 mm. Granules on the other hand are agglomerates comprising coarse and/or small fine particles, commonly produced for use in tablets and capsules. Tablets imply compacts produced for medical use that are normally of a shape that is cylindrical, with flat or convex end top and bottom surfaces, e.g., the compacts of FIGS. 1 to 3 with flat top and bottom surfaces. More preferably, the compacts are dispersible tablets. The compacts could also be used to formulate secondary products comprising other water-soluble dosage forms such as tablets, capsule, liquids, ointments, or the like, with varying physicochemical properties. The compacts may be configured for disintegration or release of turmeric and/or ginger within 0.0011 to 0.17, 0.09 to 0.5, or 0.067 to 49 hours, typically, within 0.0013 to 0.25 hour, more typically within, e.g., 0.025 to 1, 0.5 to 1.5, 1 to 3, or 0.25 to 5 minutes, in an aqueous liquid within a temperature range of 0 to 50, 40 to 60, 45 to 90, or 55 to 110° C. at ambient atmospheric pressure of no more than 1.4 atmosphere. more preferably a temperature range of 34.5 to 36, 35.1 to 35.9, 36.5 to 37, or 37.5 to 39.4° C. at ambient atmospheric pressure.

The inventive compacts may comprise a solid dosage form that may be administered orally, for example as a dispersing tablet such as an orodispersible tablet, tablets that can be swallowed whole, or tablets that are dissolved in an aqueous medium prior to administration. Published studies have authenticated the therapeutic efficacy (ability to treat a medical condition) of turmeric and ginger including Ungphaiboon et al., 2005, Hutchins-Wolfbrandt and Mistry, 2011, Naghsh, 2015, Dissanayake, Waliwita and Liyanage, 2020. Therapeutic properties of turmeric include antioxidative properties allowing for its usefulness in diabetes mellitus, inflammation and ulcer conditions. Other properties of turmeric reported include, e.g., anti-neoplastic, anticoagulant, hepatoprotective and antifungal properties. Ginger has been shown to have antinociceptive, anticancer, nephroprotective, antiviral, analgesic, antioxidant, and anti-inflammatory properties along with antitussive properties. Prior studies from literature have shown that both turmeric and/or ginger are safe for administration. Inventive compacts comprising turmeric and/or ginger may therefore have a synergistic potentiating effect for treating any of the aforementioned conditions and others. Examples of possible administration of the present invention, comprising turmeric and/or ginger, may include coadministration with vaccines administered for the novel corona virus SARS-COV2(COVID) vaccine to reduce the incidence of possible side effects including pain, inflammation and thrombosis. The compacts, comprising turmeric and/or ginger may also be administered on their own to possibly aid in treating COVID. Turmeric containing inventive compacts may similarly be co-administered with medications that present high risks of kidney damage, to counteract these risks due to turmeric's hepatoprotective properties. Like those comprising turmeric, ginger containing compacts may also be incorporated into pre-existing treatment plans used to treat the COVID and other ailments such as the common cold. asthma and the flu. Coadministration should only be made possible or promoted once studies have confirmed that there are no drug interactions that may result in adverse side effects. Users of inventive compacts may therefore experience 0 to 100% relief, e.g., at least 1, 2.5, 5, 7.5, 10, 15, 25, 33, 50, 62.5, 75, 85, 90, or 95% and/or up to 100, 99.5, 99, 97.5, 95, 92.5, 90, 85, 80, 75, 62.5, or 50% relief, when provided with a therapeutically effective amount of turmeric and/or ginger in the inventive composition. Examples of therapeutically effective amount of turmeric and/or ginger, according to literature, may be at least 1.6 to 3.2, 15 to 18, 20 to 25, 24 to 30, or 29 to 560, 70 to 140, or 25 to 1,000 mg of turmeric per kg body mass and/or at least, e.g., 0.3 to 3, 0.5 to 1, 0.65 to 0.7. 1.25 to 2, or 1.5 and/or up to 5 g of ginger per day. The weight percentage of the compact invention intended for therapeutic and/or functional food application may comprise 90.0 to 95, 92 to 97, 98 to 106, or 105 to 110 wt. % of the total target mass of the compact according to the mass range limits prescribed by the United States Pharmacopeia for dietary supplements.

Inventive compacts in the morphological form of a tablet, pellet, and/or granule, e.g., a solid dosage form, may comprise 1 to 12 layers consisting of starch, turmeric, and/or ginger, wherein a release constituent pattern of the compact may induce a 0.05 to 100% therapeutic efficacy for disorders of structure or function in mammals including pain, inflammation, and/or nausea, and wherein the constituent release pattern may initiate a minimum of one therapeutic response in a sequential manner.

Inventive compacts may be placebos configured to disintegrate in water, under ambient conditions, in no more than 8 hours. The compacts may comprise the starch (the dasheen starch, yam starch, and/or the Irish potato starch) of at least, e.g., 0.45, 0.5, 1, 20, 36, or 70 wt. %, and/or up to 100, 95, 80, 66, 52, 49, 36, 23, 20, or 15 wt. %, relative to the mass of one layer, two layers, three layers, etc., wherein inducing direct compression binding of at least one of the constituents requires subjecting the starch to compressive stress.

The compacted mixture of the inventive compacts may comprise a condiment suitable for a food and/or a beverage. Each inventive compact may be a pellet, a granule, and/or a tablet. The constituents of the compacts are released once the compact establishes contact with an aqueous liquid. This can allow for a faster preparation time for a food and/or beverage. This may result in, e.g., flavour, colour, or both being added to the final product or the improved shelf life of the product. The final product may also be classified as a functional food. The compacts may comprise the (i) turmeric, (ii) ginger, or a mixture of any (iii) two, or (iii) three of turmeric, ginger or starch, in at least 1, 2 3, 4, 7, 8, or 11 layers and/or up to 12, 10, 9, 8, 5, 4, 3, or 2 layers, with each layer being of a thickness, at the perimeter/circumference and/or centre of the compact and/or in a longest dimension in a orthogonal plane to a pressing direction, in a range of from, e.g., 0.002, 0.004, 0.05, 0.6, 0.8, 0.95, 1, 2, 3, 4.8, 5, or 8.5 cm and/or up to 9, 8, 7, 6.5, 6.72, 5.5, 4.7, 3, or 2 cm, when measured horizontally, vertically, and/or diagonally.

The inventive compacts may comprise one or multiple layers, e.g., a first layer, a second layer, a third layer, a fourth layer, etc., up to, e.g., 6, 8, 10, or 12 layers, each comprising (i) up to 100 wt. % of the turmeric, (ii) up to 100 wt. % of the ginger, (iii) up to 100 wt. % of the starch. (iv) up to 100 wt. % of a mixture of any two, or (v) up to 100 wt. % of a mixture of all three, relative to a total layer mass of at least in a range of from, e.g., 0.025 to 1.7 g, including at least 0.025, 0.027, 0.035, 0.0.39, 0.056, 0.087, 0.093, 0.1, 0.5, 1, 1.05, 1.25, 1.5, 1.6, 1.62, or 1.68 g and/or up to 1.7. 1.68. 1.6. 1.5. 1.3, 1.28. 0.58, 0.5, or 0.43 g.

The inventive compacts may comprise, e.g., a first layer, second layer, and/or fourth layer, with each layer comprising a total mass of, e.g., at least 300, 315, 350, 375, 390, 400, 455, 500, 515, 600, or 850 mg and/or up to 900, 875, 850. 500, 475, or 320 mg. One layer, two layers, three layers, etc. may comprise. e.g., (i) no less than 4 wt. %, e.g., at least 4, 4.7, 5, 5.2, 6, 6.5, 7.8, or 10 wt. % and/or up to 20, 15, 10, 5, or 4.79 wt. % of the dasheen starch, and e.g., at least 50, 52.4, 55, 65, 75, 77.58, 82, 92.5, 93.2, 95 or 96 wt % and/or as high as 96, 85, 78, 70, 65, or 60 wt. % of (ii-a) the turmeric only, (ii-b) the ginger only, or (ii-c) a mixture of the turmeric and the ginger, relative to the mass of the layer. These inventive compacts may have a disintegration rate, in water at 37° C., of, e.g., 4.0, 4.56, 5, 6, 7, 7.5, 8.2, 9, 20, 30, 45 or 50 mg/s and/or up to 50, 49, 46, 40, 35, 30.1, 30, 28, 20, 9.5, 8, or 7 mg/s. Within these inventive compacts, the turmeric and/or the ginger may be suitable to functionally supplant, e.g., at least a portion of, a customary lubricant, the dasheen starch may be suitable to functionally supplant, e.g., at least a portion of, a customary disintegrant. One or both of the rhizome powders, along with the starch may also function as binders, specifically, DC binders.

The inventive compacts may comprise, e.g., a first layer, second layer, and/or fourth layer, with each layer comprising a total mass of, e.g., at least 300, 315, 350, 375, 390, 400, 455, 500, 515, 600, or 850 mg and/or up to 900, 875, 850, 500, 475, or 320 mg, wherein, relative to a total mass of each layer, a first and second layer comprises the dasheen starch, at 100 wt. %; and a third layer comprising the turmeric and/or the ginger of a wt. % of at least, e.g., 50, 52.4, 55, 65, 75, 77.58, 82, 92.5, 93.2, 95 or 96 wt % and/or as high as 96, 85, 78, 70, 65, or 60 wt. %, wherein the layers are arranged in a stacked pattern and can be differentiated according to the contents, e.g., (i) starch-the turmeric-starch, (ii) starch-the turmeric and the ginger-starch, (iii) starch-starch and the turmeric-starch, (iv) starch-starch, the turmeric and the ginger-starch, and/or (v) starch-the ginger-starch. The structural design of the compact may be, e.g., like that illustrated in FIG. 3. The disintegration rate of at least one layer is, e.g., 0.4, 0.5, 0.76, 0.85, 1, 3.5, 3.47 or 4.4 mg/s and/or up to 4.5, 4.4, 3.9, 2.5, 1, 1.7, 1.45, 0.95, 0.65, or 0.4 mg/s.

The inventive compacts may comprise, e.g., a first layer, second layer, and/or fourth layer, with each layer comprising a total mass of, e.g., at least 300, 315, 350, 375, 390, 400, 455, 500, 515, 600, or 850 mg and/or up to 900, 875, 850, 500, 475, or 320 mg, wherein, relative to a total mass of each layer, a first and second layer comprises the turmeric, at 100 wt. %; and a third layer comprising the ginger of a wt. % of at least, e.g., 90, 90.3, 90.5, 92, 93.2, 95.4, 97.1, 97.5, 98.8, 99, or 99.9 wt % and/or as high as 100, 98, 97.5, 96, 95.5, or 91.6 wt. %. wherein the layers are arranged in a stacked pattern with the layer comprising ginger being centralised. The structural design of the compact may be, e.g., as illustrated in FIG. 3. The disintegration rate of the compact is, e.g., at least 7, 5, 8.2, 8.5, 10, 11, 12, 13, 13.3, 13.8, 14.3, 15.5, 16, 16.7, 16.93 mg/s and/or up to 17, 16, 12, 10.5, 10, 9.8, 8.5, or 7 mg/s.

The ginger and the turmeric have been reported in literature to have properties to promote product preservation. The ginger and/or the turmeric may therefore supplant, at least a portion of, a preservative in the inventive compacts, or secondary product comprising the inventive compacts. The inventive compacts may further comprise a customary preservative, e.g., sodium benzoate, to extend, or partake in extending. the shelf life of the invention and/or a secondary product of which the inventive compacts are constituents.

Investigations have identified that the turmeric and/or the ginger, once subjected to compressional pressure application, comprises a functionality typical of a direct compressional binder, with the possible exception of being inert, thereby promoting cohesiveness, or cohesiveness and adhesiveness of constituents of the compact. Here cohesiveness means like parts (or parts of one constituent), within the compact, binding to themselves, e.g., binding taking place between particles of turmeric powder, and adhesiveness implies constituents that differ in composition binding to each other, e.g., particles of turmeric powder binding to particles of another natural constituent and/or synthetic constituent. The study also showed that turmeric and/or ginger may have higher compressibility properties. compared to that of pharmaceutical actives of solid dosage forms wherein the composition normally comprises no more than 25 wt. % of the total mass of the dosage form. Investigations also revealed that the compact release rate may have direct correlations with the wt. % of the turmeric, the region of incorporation of the turmeric, and/or the friability of the compact. A one-year study conducted with compacts comprising the turmeric only, revealed surprising direct correlations between release rates and storage time. Data also revealed that inverse correlations may exist between compact rate of release and the wt. % content of ginger. It was therefore concluded that turmeric appears to exhibit superior binding properties resulting in increased disintegration time coupled with reduced friability values, relative to the ginger, in one or more compacts of the present invention. It was also concluded that the turmeric may be added to a layer that may result in an increase in the plasticity or plastic deformation of that layer. Investigations involving compacts comprising turmeric and/or ginger also showed multi-layered stacked compacts, similar to those illustrated in FIGS. 2 to 3, being unstable with pre-compressed layers not remaining adhered to consecutive layers during storage, e.g., an inventive compact produced by initially preparing a first compact comprising of the turmeric or the ginger and not removing it from the die, followed by the generation of a second multi-layered compact, within the said die, of which the first compact is a constituent. For inventive compacts with stacked layers to be formed that will remain adhered (without layers separating after compact formation) to adjacent surrounding layers, in storage, the powder raw materials all have to be added in the respective desired sequential order then compressed all at once to achieve a stable compact. For inventive compacts with embedded layers, e.g., those illustrated in FIGS. 5 to 9, layer separation may occur if one or more of the embedded layers are produced as compacts first; this would result in larger spacing lengths between surface peripheries of layers, which could be an added feature of the compact invention, e.g. to promote faster disintegration and/or dissolution.

Comparative studies also lead to the discovery of a possible direct correlation between the rate of disintegration of one or more layers, and the homogeneity of a constituent mix of one or more layers comprising dasheen starch in its natural state in no more than a range of from 1 to 40 wt. % and the turmeric and/or the ginger in at least a range of from 50 to 96 wt. %, of the total mass of a layer and/or the compact. Comparisons were made among three compact types, each comprising equal amounts of the turmeric. A compact wherein the starch was added first and last to the die of the tablet tooling, to constitute a segregated mix allowing for the formation of the stacked layers of a compact with upper and lower layers comprising only the starch (compact 1, e.g., arranged as seen in FIG. 3), was compared to two other compact types, one comprising only one layer constituting only the turmeric (compact 2, e.g., arranged as seen in FIG. 1), and the other comprising only one layer comprising a one in six mixture of the starch and the turmeric that was mixed, using the tumbling method, and assumed to be homogenously mixed (compact 3, e.g., arranged as seen in FIG. 1). The disintegration rate of each layer comprising turmeric were compared and found to be in the order of compact 1 to compact 3. The disintegration rate of the layer comprising the turmeric of compact 3 was at least in a range of from 4.56 to 50 mg/s, and that of compact 1 was at least in a range of from 1.19 to 2.76 mg/s, with disintegration generally occurring within no more than 8 minutes. In addition, assessments of compact 1 with varying masses of the starch of 100 wt. % of the mass of the outer layers, showed a direct correlation between the mass of starch and the disintegration rate of the inner layer of the turmeric. Similar results are expected. for example, if the layer of the turmeric of each compact type, were alternately, of the ginger, or a mixture of the ginger and the turmeric.

Other studies also indicated that inventive compacts of 100% of the starch may have hardness values above 20 kg. This was indicated with maximum diametral compression application (application of 20 kg) with no compact fracture being detected.

Other comparative studies indicated that with the addition of the turmeric in at least a range of from 60 to 100 wt. % of the total mass of a compact with the dasheen starch being its only other constituent of the homogenously mixed mixture, produced using the tumbling method, there may be significantly less difficulty experienced in ejecting the compact from the die relative to a compact comprising 100 wt. % of the starch, of the total mass of the compact. The presence of the turmeric may thereby eliminate the need for the application, or a portion thereof, of a customary lubricant.

For determining hardness and friability of inventive compacts herein, raw materials and compact masses were weighed accurately using a Ax423 Adventurer Balance 420 g×1 mg. Examples of the inventive compacts were produced, using a Carver Manual Press 3850, with slow manual application of pressure at a constant rate, and customised tablet tooling (upper and lower punches, and die) produced and sold by Natoli Engineering Company. Hardness of the inventive compacts were determined. using a LIH-1 Tablet Hardness Tester, at ambient temperature, by placing one compact at a time between a fixed jaw and a movable jaw, slowly applying diametral compression at a constant rate until the compact fractures. The LIH-1 Tablet Hardness Tester beeps and provides a reading in kilogram once the compact has fractured. Friability tests were carried out with sample batches of exemplary inventive compacts that weighed as close as possible to 6.5 g, using a LIC-1 Tablet Friability Tester, by tumbling each batch at 100 rpm for 25 cycles per minute, and thereafter determining mass loss percentage due to abrasion or fracture of the compacts in each batch, after dedusting if necessary. Disintegration time for the inventive compacts and/or one or more layers of the inventive compacts, was carried out using a LIJ-3 Disintegration Tester by placing six compacts at a time in a basket rack assembly. Once there was complete breakdown to allow for constituents to pass through the screen of the basket rack, disintegration is said to have occurred and the end time recorded. The medium used for disintegration was water which was maintained at a constant temperature of 37±2° C. Compact thickness can be determined using a Outside Premium Precision Machinist Tool micrometer.

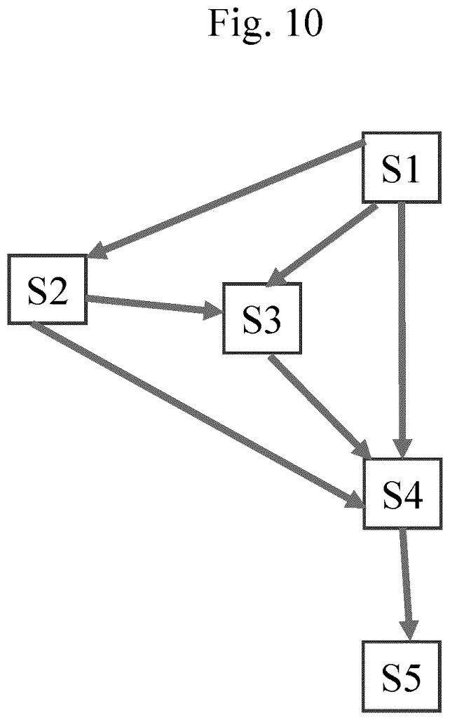

The preferred method of preparation of the present invention, after optionally sanitising initially, mainly comprises air drying, milling, and lastly content compression by DC. Prior to compact production, a homogenously mixed mixture may be prepared using the tumbling method for a time of no less than 2 seconds. This mixture can then be added to the die for compact production with compressional stress. Alternately a segregated mixture may be prepared by adding the constituents to a tablet die, in a sequential order, forming the segregated mixture that allows for one method for the production of multi-layered compacts with compressive stress. The process of production of the inventive compacts may include those illustrated in the schematic representation of several exemplary methods of manufacturing within the scope of the invention seen in FIG. 10. Each step is represented by the letter ‘S’ followed by an Arabic number, e.g. S3. The numbering sequence is not an indication of the sequential order of the steps of manufacturing; for example S1, S2, S4, S5 then S6 is one method of manufacturing compacts of the present invention. S1 involves adding constituents to a die allowing for at least one layer to be formed. S2 represents a step within which the constituents, of one or more of the layers, undergo wet and/or dry granulation to form final product compacts, and/or compact constituents with a granule morphology. S3 involves constituents, for one or more of the layers to be formed, being subjected to compressional pressure with the lowering of an upper punch on to the constituents within the die to induce the binding functionality of the turmeric, the ginger and/or the starch, and allowing adequate dwell time for the formation of compact constituents with a pellet and or tablet morphology, before the further addition of constituents. S4 involves inducing and/or further inducing the binding functionality of the turmeric, the ginger and/or the starch constituents for the formation of a final product compact, e.g., a first compact, a second compact and/or a third compact. For the inventive compacts, a first compact is the final product from the production process of S1 to S2 (method 1) and/or S1 to S4 to S5 (method 2); or a second compact is the final product from the production process of S1 to S2 to S4 to S5 (method 3), and/or S1 to S3 to S4 to S5 (method 4), wherein the second compact houses a first compact; or a third compact is the final product, produced by the manufacturing process of S1 to S2 to S3 to S4 to S5 (method 5), wherein the first compact is a constituent of the second compact and the second compact, housing the first, is a constituent of the third compact. Lastly S5 involves ejecting the compact from the die. Examples of these methods of producing the inventive compacts include, but are not limited to, applying compressional pressure, with, e.g., an upper limit of 10 metric tonnes, e.g., 0.15, 0.3, 1, 2, 3, 4, 5, 6.5, 7.5, 8, etc. to 10 metric tonnes. for a dwell time of at least 8 to 300 seconds, to constituents in the die, comprising (i) the turmeric, (ii) the ginger, (iii) the starch. or (iv-a) a mixture, e.g. segregated or homogenously mixed, comprising any two or (iv-b) all three, of constituents (i) to (iii), wherein the pressure is sufficient to induce a direct compression binding functionality of the turmeric, the ginger and/or the starch. Compact ejection from the die may follow. after which the compact may undergo expansion, e.g. diametral expansion of a circular compact. Optionally, further processing can take place resulting in the formation of, e.g., a secondary compact, e.g., constituting a compact from method 1 a tertiary compact, e.g., constituting a compact from method 3, a quaternary compact, e.g., constituting a compact from method 5, etc., any of which may be the final compact product. Further processing may also imply or include. e.g., (i) adding further constituents to the die, after compressional pressure was applied to previously added constituents to form a compact that was not ejected from the die, and subsequently applying compressional pressure to induce and/or further induce binding functionality of the starch, the turmeric and/or the ginger. thereby forming a secondary compact to that of the constituent compact, of which the constituent compact may also be the product of at least one compact.

Further processing may also imply. after removing the compact from the die, further constituents, and/or additional portions of the constituents, may be added to a second die comprising a larger cavity and further applying compressional pressure to induce and/or further induce binding functionality of the starch, the turmeric and/or the ginger, thereby forming a secondary compact to that of at least one constituent compact that may be of a morphological form inclusive of a pellet, granule and/or tablet.

### EXAMPLES

The following are examples of inventive compacts, in the scope of the present

invention. It is to be understood, however, that several other variations and modifications can be formulated which will not deviate significantly from the scope of the present invention.

### Compact Production

The 100% natural compact preparation examples produced by DC, within the scope of the present invention, were based on generating dispersible compacts that could disintegrate within 3 minutes, e.g., dispersible dosage form tablets, comprising the ginger only or the turmeric and the ginger, (i) without the need for the addition of a lubricant, a disintegrant and/or a binder, (ii) that were easy to eject from the die, and (iii) that had a friability below 1%.

### Example 1

95 to 100 wt. %±5 wt. % ginger powder, based on the total mass of the compact, was compressed into 500 mg compacts that disintegrated within 0.2 to 0.5 minutes and has a friability between 0.6 to 0.8%.

### Example 2

A three layered 750 mg compact was prepared by sequentially adding turmeric powder, ginger powder and further adding turmeric powder at 95 to 100 wt. %±5 wt. % to the die, based on the total mass of each layer, that were of equal amounts. The compact had an average disintegration time and friability not significantly different from one layered compacts prepared from 95 to 100 wt. %±5 wt. %, relative to a compact mass of 500 mg of turmeric powder, that had a disintegration time of within 1 to 2 minutes and friability values between 0.1 to 0.3%. Similarly to the compacts comprising turmeric only, the three layered compacts were easily ejected from the die.

1. A compact, comprising:
turmeric and/or ginger,
wherein the turmeric or the ginger is suitable to functionally supplant at least a portion of customary adjunct,
wherein the compact comprises a first layer,
wherein the compact is a granule, pellet, or tablet,
wherein the compact has a total mass upper limit of 13 g,
wherein each layer has a total mass upper limit of 13 g,
wherein each layer is positioned at a 0 to 90° angle from an orthogonal plane to a pressing direction,
wherein the compact has a thickness in a longest dimension in the orthogonal plane in a range of from 0.0001 to 12 cm,
wherein each layer has a layer thickness in a longest dimension in the orthogonal plane in a range of from 0.0001 to 12 cm,
wherein the compact friability has an upper limit of 7%,
wherein the compact is configured to release its constituents in an aqueous environment,
wherein the compact is configured to disintegrate within 0.00011 to 0.5 hours, at a temperature in a range of from 0 to 100° C., at ambient atmospheric pressure, and
wherein the compact is configured such that a limiting factor for disintegration of the compact is disintegration of at least a first layer.

2. The compact of claim 1, wherein a starch is present, in its native state, and wherein the starch comprises dasheen starch.

3. The compact of claim 1, further comprises:
a second layer; and
a space between the first layer and the second layer,
wherein the first and second layers are differentiated by thickness, shape, and/or colour,
wherein the space comprises air and/or a barrier layer, and
wherein the layers are spaced by a spacing length in a range of from at least 0.00001 to 120 mm.

4. The compact of claim 1, wherein the first layer encompasses a second layer.

5. The compact of claim 1, wherein the turmeric is suitable to functionally supplant at least a portion of a direct compression binder, and
wherein the turmeric contributes to the compact having a hardness lower limit of 0.4 kg and a friability upper limit of 4.5%.

6. The compact of claim 1, wherein the ginger is suitable to functionally supplant at least a portion of a direct compression binder, and
wherein the ginger contributes to the compact having a hardness lower limit of 0.4 kg and a friability upper limit of 6.9%.

7. The compact of claim 1, comprising:
no customary binder, disintegrant, or lubricant.

8. The compact of claim 1, further comprising:
a second layer, comprising the turmeric at 100 wt. %, relative to a total mass of the second layer, and
a third layer, comprising the ginger in a range of from 50 to 100 wt. %, relative to a total mass of the third layer,
wherein the disintegration rate of the compact in water, at a temperature in a range of 34.5 to 39.4° C., is in a range of from 0.4 to 17 mg/s,
wherein the compact has a mass in a range of from 50 to 900 mg.

9. The compact of claim 1, further comprising:
a second layer, comprising the turmeric at 100 wt. %, relative to a total mass of the second layer, and
a third layer, comprising a mixture of the turmeric and the starch in a range of from 50 to 100 wt. %, relative to a total mass of the third layer,
wherein the disintegration rate of the compact in water, at a temperature in a range of 34.5 to 39.4° C., is in a range of from 0.4 to 17 mg/s.
wherein the compact has a mass in a range of from 50 to 900 mg.

10. The compact of claim 1, further comprising:
a second layer, comprising the starch as dasheen starch, at 100 wt. %, relative to a total mass of the second layer, and
a third layer, comprising the turmeric in a range of from 50 to 100 wt. %, relative to a total mass of the third layer,
wherein the disintegration rate of the compact in water, at a temperature in a range of 34.5 to 39.4° C., is in a range of from 0.4 to 17 mg/s,
wherein the compact has a mass in a range of from 50 to 900 mg.

11. The compact of claim 1, further comprising:
a second layer, comprising the starch as dasheen starch, at 100 wt. %, relative to a total mass of the second layer, and
a third layer, comprising the ginger in a range of from 50 to 100 wt. %, relative to a total mass of the third layer,
wherein the disintegration rate of the compact in water, at a temperature in a range of 34.5 to 39.4° C., is in a range of from 0.4 to 17 mg/s,
wherein the compact has a mass in a range of from 50 to 900 mg.

12. The compact of claim 1, further comprising:
a second layer, comprising a mixture of (i) the turmeric and (ii-a) the starch or (ii-b) the ginger, at 100 wt. % relative to a total mass of the second layer, and
a third layer, comprising the ginger at 100 wt. %, relative to a total mass of the third layer,
wherein the disintegration rate of the compact in water, at a temperature in a range of 34.5 to 39.4°° C., is in a range of from 0.4 to 17 mg/s,
wherein the compact has a mass in a range of from 50 to 900 mg.

13. The compact of claim 1, further comprising:
a second layer, comprising a mixture of the turmeric, the starch and the ginger, at 100 wt. % relative to a total mass of the second layer, and
a third layer, comprising the ginger at 100 wt. %, relative to a total mass of the third layer,
wherein the disintegration rate of the compact in water, at a temperature in a range of 34.5 to 39.4° C., is in a range of from 0.4 to 17 mg/s,
wherein the compact has a mass in a range of from 50 to 900 mg.

14. The compact of claim 1, further comprising:
a second layer, comprising a mixture of the ginger and the starch, at 100 wt. % relative to a total mass of the second layer, and
a third layer, comprising the ginger in a range of from 50 to 100 wt. %, relative to a total mass of the third layer,
wherein the disintegration rate of the compact in water, at a temperature in a range of 34.5 to 39.4° C., is in a range of from 0.4 to 17 mg/s, and
wherein the compact has a mass in a range of from 50 to 900 mg.

15. The compact of claim 1. further comprising:
sodium benzoate.

16. The compact of claim 1, wherein the compact is configured to induce a first therapeutic response.
wherein the compact is configured such that a limiting factor for a therapeutic response is a disintegration rate of at least a first layer.

17. The compact of claim 1, wherein the compact is a dosage form. wherein the turmeric is present and at least supplants a portion of the functionality of an active,
wherein the longest dimension of the compact is in a range of from 0.002 to 4 cm, and
wherein the compact has a mass in a range of from 0.0001 to 2 g.

18. The compact of claim 1, wherein the compact is a dosage form, wherein the ginger is present and at least supplants a portion of the functionality of an active,
wherein the longest dimension of the compact is in a range of from 0.002 to 4 cm, and
wherein the compact has a mass in a range of from 0.0001 to 2 g.

19. The compact of claim 1, wherein the compact is a condiment, wherein the turmeric is present,
wherein the longest dimension of the compact is a range of from 0.002 to 9 cm, and
wherein the compact has a mass in a range of from 0.0001 to 13 g.

20. The compact of claim 1, wherein the compact is a condiment, wherein the ginger is present,
wherein the longest dimension of the compact is a range of from 0.002 to 9 cm, and
wherein the compact has a mass in a range of from 0.0001 to 13 g.
